(19) United States
(12) Patent Application Publication
(10) Pub. No.: US 2025/0002894 A1
(43) Pub. Date: Jan. 2, 2025

(54) BIOCATALYTICAL COMPOSITIONS

(71) Applicant: Inofea AG, Muttenz (CH)

(72) Inventors: Rita CORRERO, Hegenheim (FR); Carolina GIUNTA, Basel (CH); Lars GROSSNIKLAUS, Basel (CH); Anna WEBER, Grenzach-Wyhlen (DE); Manon BRIAND, Ennetbaden (CH)

(21) Appl. No.: 18/709,228

(22) PCT Filed: Nov. 17, 2022
(86) PCT No.: PCT/EP2022/082288

§ 371 Date: May 10, 2024

(30) Foreign Application Priority Data
Nov. 18, 2021 (EP) 21209017.9

(51) Int. Cl. C12N 11/14; C12N 9/20

(52) U.S. Cl. CPC C12N 11/14; C12N 9/20; C12Y 301/01003

(57) ABSTRACT

A composition comprising a solid carrier, wherein a surface of the solid carrier comprises an organosilane with a proviso that the organosilane is not an aminosilane, or wherein the surface of the solid carrier comprises at least two organosilanes which are different from each other; a functional constituent; and a protective layer to protect the functional constituent. A method of producing the composition and uses of the composition.

### CROSS-REFERENCE TO RELATED APPLICATIONS

The present application is the U.S. National Phase of PCT/EP2022/082288, filed on 17 Nov. 2024, which claims priority to European Patent Application No. 21209017.9, filed on 18 Nov. 2021, the entire contents of which are incorporated herein by reference.

### THE FIELD OF THE INVENTION

The present invention relates to a composition comprising a solid carrier, wherein a surface of the solid carrier comprises an organosilane with a proviso that the organosilane is not an aminosilane, or wherein the surface of the solid carrier comprises at least two organosilanes which are different from each other; a functional constituent; and a protective layer to protect the functional constituent. The present invention also relates to a method of producing the composition and uses of the composition.

### BACKGROUND OF THE INVENTION

Proteins such as enzymes are frequently needed, e.g., in industrial applications, diagnostics or for therapeutic use. In order to stabilize the proteins and/or to provide resistance to various types of stresses it has been suggested in the prior art to immobilize the proteins on the surface of a carrier and to protect them with a layer of protective material. Such an approach has been described, e.g., in WO2015/014888 which discloses a biocatalytical composition comprising a solid carrier, a functional constituent like an enzyme and a protective layer for protecting the functional constituent by embedding the functional constituent at least partially and a process to produce such biocatalytical composition. Nevertheless there is a need for providing an economical and easy-to-use system for protecting functional constituents like proteins or protein-type compounds, particularly enzymes, against unfavorable influences thereby maintaining or even increasing their biological activity. Thus, there is a need for a protection system, which allows for use of a protected protein or enzyme at high enzymatic activity.

### SUMMARY OF THE INVENTION

The present invention provides a composition comprising a solid carrier, wherein the surface of the solid carrier comprises an organosilane with the proviso that the organosilane is not an aminosilane, or wherein the surface of the solid carrier comprises at least two organosilanes which are different from each other; a functional constituent; and a protective layer to protect the functional constituent. The present invention further provides a method of producing a composition, the composition comprising a solid carrier, wherein the surface of the solid carrier comprises an organosilane with the proviso that the organosilane is not an aminosilane, or wherein the surface of the solid carrier comprises at least two organosilanes which are different from each other; a functional constituent; and a protective layer to protect the functional constituent, the method comprising the following steps:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) optionally re-suspending the solid carrier comprising the functional constituent protected by the protective layer in an organic solvent; and
(g) isolating the solid carrier comprising the functional constituent protected by the protective layer from the organic solvent suspension.

It has been surprisingly found that the activity of the immobilized enzymes as described in WO2015/014888 can be significantly increased by modifying the surface of the solid carrier with an organosilane other than 3-aminopropyltriethoxysilane or with at least two different organosilanes prior to immobilising the enzyme.

### BRIEF DESCRIPTION OF THE FIGURES

FIG. 1 shows immobilization of CalB on modified silica particles. Silica particles were modified with 3-aminopropyltriethoxysilane (APTES) following the procedure as described in WO2015/014888 or with APTES and Benzyl-triethoxysilane according to the invention. The catalytic activity of the CalB was measured in an assay using para-nitrophenyl butyrate as substrate (p-NPB). The recovered activity of the CalB immobilized in surface modified particles was 6 times higher than the one immobilized on APTES modified particles.

FIG. 2 shows activity of shielded CalB immobilized on modified silica particles. Silica particles were modified with APTES following the procedure as described in WO2015/014888 or with APTES and Benzyl-triethoxysilane according to the invention. The shielding with APTES and TEOS was performed following the procedure as described in Example 1 of WO2015/014888. The catalytic activity of the shielded CalB was measured in an assay using para-nitrophenyl butyrate as substrate (p-NPB). The activities of the CalB immobilized on a carrier modified with APTES only or with APTES and benzyl-triethoxysilane and shielded with a layer of APTES and TEOS were 7 U/g_{carrier} and 10 U/g_{carrier}, respectively.

FIG. 3 shows immobilization of a model enzyme (CalB) on modified silica particles. Silica particles were modified with different ratios of GPTMS and n-octyl-triethoxysilane (Octyl). The catalytic activity of the CalB was measured in an assay using para-nitrophenyl butyrate as substrate (p-NPB). The recovered activity of the CalB immobilized in particles modified with GPTMS and Octyl with molar ratio 4.5×10⁻⁶ mmol/1.8×10⁻⁵ mmol was 14 times higher than the activity recovered on particles not modified and 3 times higher than particles modified with APTES only.

FIG. 4 shows activity of shielded CalB immobilized on modified silica particles. Silica particles were modified with 5.3×10⁻⁴ mmol of (3-glycidyl-oxypropyl)-trimethoxysilane (GPTMS). The enzymatic activity recovered in particles modified with 5.3×10⁻⁴ mmol of GPTMS was 97.5 U/g_{carrier}, while the activity recovered on particles not treated was 65 U/g_{carrier} meaning 1.5 times lower than the activity recovered in particles modified with GPTMS.

### DETAILED DESCRIPTION OF THE INVENTION

The present invention relates to a composition comprising a solid carrier, wherein a surface of the solid carrier comprises an organosilane with a proviso that the organosilane is not an aminosilane, or wherein the surface of the solid carrier comprises at least two organosilanes which are different from each other; and a protective layer to protect the functional constituent.

The present invention also relates to a method of producing the composition, the method comprising the following steps:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) optionally re-suspending the solid carrier comprising the functional constituent protected by the protective layer in an organic solvent; and
(g) isolating the solid carrier comprising the functional constituent protected by the protective layer from the organic solvent suspension.

For the purposes of interpreting this specification, the following definitions will apply and whenever appropriate, terms used in the singular will also include the plural and vice versa.

It is to be understood that the terminology used herein is for the purpose of describing particular embodiments only and is not intended to be limiting.

The term “solid carrier” as used herein refers to a particle. Usually the solid carrier is a monodisperse particle or a polydisperse particle, preferably a monodisperse particle. The solid carrier usually comprises organic particles, inorganic particles, organic-inorganic particles, self-assembled organic particles, silica particles, gold particles, magnetic particles and titanium particles. The particle size of the solid carrier is usually between 1000 μm and 1 nm, preferably between 100 μm and 10 nm, particularly between 50 μm and 50 nm, more particularly between 1 μm and 100 nm.

The term “organosilane” as used herein refers to monomeric silicone-based chemicals, similar to hydrocarbons, which have at least one direct bond between a silicon atom and a carbon atom in the molecule. Various organosilanes are known in the art, including but not limited to organosilanes carrying a functional polar group selected from an alcohol, an amine, a carboxylate, a thiol, a thioether, a guanidinium, an amide; organosilanes selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane; or organosilanes selected from the group consisting of benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane (GPTMS), and n-octyl-triethoxysilane. An organosilane used in the present invention may carry a functional binding group, such as an epoxy group, which allows e.g. covalent binding of the organosilane to the functional constituent.

The term “an organosilane”, e.g., the term “wherein the surface of the solid carrier comprises an organosilane” means that the surface of the solid carrier comprises a multitude of the same organosilane, e.g, if the oganosilane is (3-Glycidyl-oxypropyl)-trimethoxysilane then the surface of the solid carrier comprises a multitude of (3-Glycidyl-oxypropyl)-trimethoxysilane.

The term “at least two organosilanes which are different from each other”, e.g., the term “wherein the surface of the solid carrier comprises at least two organosilanes which are different from each other” means that the surface of the solid carrier comprises a multitude of at least two different organosilanes, e.g., if the first organosilane is APTES and the second organosilane is benzyl-triethoxysilane, then the surface of the solid carrier comprises a multitude of APTES and a multitude of benzyl-triethoxysilane. The surface of the solid carrier is usually partially or fully, preferably partially, covered by the organosilane or the at least two organosilanes.

The term “hydrophilic organosilane” as used herein refers to organosilanes carrying a functional polar group such as a functional polar group selected from an alcohol, an amine, a carboxylate, a thiol, a thioether, a guanidinium, an amide; and includes organosilanes selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxynethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane.

The term “hydrophobic organosilane” as used herein refers to organosilanes carrying a functional non-polar group such as, e.g., a functional non-polar group selected from: continuous carbon chain and hydrocarbon functional groups such as alkanes, alkenes, alkynes and arenes and includes organosilanes selected from the group consisting of benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane, and n-octyl-triethoxysilane.

The term “functional constituent” as used herein refers to a constituent which imparts to the solid carrier to which this functional constituent is added its characteristic, functional property. A functional constituent in the sense of the present invention is usually a protein, e.g., an enzyme, an antibody, or RNA which has catalytic activity. Thus, in case the “functional constituent” is an enzyme, which is the preferred functional constituent, the carriers comprising the enzyme are enzymatically active. The functional constituent is immobilized to the solid carrier. Preferably the functional constituent is covalently bound to the solid carrier, in particular, it is covalently bound to the surface of the solid carrier.

The term “linker” as used herein refers to any linking reagents containing reactive ends, which are capable of binding to specific functional groups (e.g., primary amines, sulfhydryls, etc.) of the solid carrier and the functional constituent, respectively. Various types of linkers are known in the art, including but not limited to straight or branched-chain carbon linkers and polyether linkers. For example, a linker may be immobilized on a solid carrier and then the functional constituent may be bound to an unoccupied binding-site of the linker. Alternatively, the linker may firstly bind to the functional constituent and then the linker bound to the functional constituent may bind with its unoccupied binding-site to the organosilanes comprised by surface of the solid carrier.

The term “protective layer” as used herein refers to a layer for protecting the functional constituent of the composition. The protective layer of the present invention is usually built with building blocks at least part of which are monomers capable of interacting with both each other and the immobilized functional protein. The protective layers are usually homogeneous layers where all functional constituents, e.g., all enzyme present in the protective layer is active in the same way. The protective layer covers fully the solid carrier and covers partially or fully the functional constituent. Thus the functional constituent is partially or fully embedded by the protective layer.

The term “partially embedded functional constituent” as used herein shall mean that the functional constituent, e.g., the protein is not fully covered by the protective layer, thus, the functional constituent is not fully embedded in the protective layer. In one embodiment less than 50% of the functional constituent of interest are covered by the protective layer, though typically more at least 70% will be covered, thus improving protection of the functional protein. In a particularly preferred embodiment, at least 70%, particularly at least 80%, more particularly at least 90%, most particularly at least 95% of the functional constituent are covered by the protective layer.

The term “fully embedded functional constituent” as used herein shall mean that the functional constituent according to the invention is 100% covered by the protective layer, i.e., that also the active site is covered.

The term“organic solvent”, as used herein shall mean a carbon-based substance that is used to dissolve another substance or substances, i.e., is used to re-suspend the solid carrier comprising the functional constituent protected by the protective layer in step e) of the present method. Since an organic solvent is carbon-based, it always has at least one carbon atom in its chemical structure. An organic solvent will also always have at least one hydrogen atom. Organic solvents usually comprise organic polar protic solvents, organic polar aprotic solvents and organic non-polar solvents. Organic polar protic solvents are, e.g., methanol, ethanol, n-propanol, isopropanol, butanol and larger alcohols, acetic acid, formic acid. Organic polar aprotic solvents are, e.g., acetone, acetonitrile, tetrahydrofurane, dimethylformamide, pyridine. Organic non-polar solvents are, e.g., ethyl-acetate, diethyl-ether, methyl-ethyl-ketone, pentane, hexane, heptane, cyclohexane, toluene, benzene and nitrobenzene.

In a first aspect, the present invention provides a composition comprising a solid carrier, wherein the surface of the solid carrier comprises an organosilane with the proviso that the organosilane is not an aminosilane, or wherein the surface of the solid carrier comprises at least two organosilanes which are different from each other; a functional constituent; and a protective layer to protect the functional constituent.

In one embodiment, the composition comprises a solid carrier, wherein the surface of the solid carrier comprises an organosilane with the proviso that the organosilane is not an aminosilane.

In a further embodiment, the composition comprises a solid carrier, wherein the surface of the solid carrier comprises at least two organosilanes which are different from each other.

In a preferred embodiment, the present invention provides a composition comprises a solid carrier, wherein the surface of the solid carrier comprises an organosilane with the proviso that the organosilane is not an aminosilane; a functional constituent; and a protective layer to protect the functional constituent.

In a further preferred embodiment, the present invention provides a composition comprising a solid carrier, wherein the surface of the solid carrier comprises at least two organosilanes which are different from each other; a functional constituent; and a protective layer to protect the functional constituent.

In one embodiment, the organosilane or the at least two organosilanes are covalently bound to the surface of the solid carrier.

In one embodiment, the organosilane or the at least two organosilanes are selected from the group consisting of organosilanes carrying a functional polar group selected from an alcohol, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, ureidopropyltrihydroxyethoxysilane, benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane, and n-octyl-triethoxysilane.

In a further embodiment, the organosilane or the at least two organosilanes are selected from the group consisting of hydrophobic organosilanes and hydrophilic organosilanes.

In a further embodiment, the surface of the solid carrier comprises two organosilanes, wherein the first organosilane is a hydrophobic organosilane or a hydrophilic organosilane and the second organosilane is a hydrophilic organosilane, wherein the second organosilane is different from the first organosilane.

In a preferred embodiment, the surface of the solid carrier comprises two organosilanes, wherein the first organosilane is a hydrophobic organosilane and the second organosilane is a hydrophilic organosilane.

In a further preferred embodiment, the surface of the solid carrier comprises two organosilanes, wherein the first organosilane is a hydrophobic organosilane and the second organosilane is a hydrophobic organosilane, wherein the second hydrophobic organosilane is different from the first hydrophobic organosilane.

In a further more preferred embodiment, the second organosilane of the two or at least two organosilanes is 3-aminopropyltriethoxysilane (APTES).

In an even more preferred embodiment, the first organosilane of the two or at least two organosilanes is a hydrophobic organosilane selected from the group consisting of benzyl-triethoxysilane, (3-Glycidyl-oxypropyl)-trimethoxysilane and n-Octyl-triethoxysilane.

In particular preferred embodiment, the first organosilane of the two or at least two organosilanes is benzyl-triethoxysilane.

In a most preferred embodiment, the first organosilane of the two or at least two organosilanes is benzyl-triethoxysilane and the second organosilane of the two or at least two organosilanes is APTES.

In a further most preferred embodiment, the first organosilane of the two or at least two organosilanes is (3-Glycidyl-oxypropyl)-trimethoxysilane and the second organosilane of the two or at least two organosilanes is n-Octyl-triethoxysilane.

In a further embodiment, the hydrophilic organosilane is selected from the group consisting of organosilanes carrying a functional polar group selected from an alcohol, an amine, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane.

In a further embodiment, the hydrophilic organosilane is selected from the group consisting of organosilanes carrying a functional polar group selected from an alcohol, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane.

In a further embodiment, the hydrophobic organosilane is selected from the group consisting of benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane, and n-octyl-triethoxysilane.

In a further embodiment, the organosilane or the at least two organosilanes comprises at least one epoxy group. Thus in a preferred embodiment, the composition comprises a solid carrier, wherein the surface of the solid carrier comprises an organosilane or at least two organosilanes which are different from each other, wherein the organosilane or one of the at least two organosilanes which are different from each other comprise at least one epoxy group; a functional constituent; and a protective layer to protect the functional constituent. In a more preferred embodiment, the composition comprises a solid carrier, wherein the surface of the solid carrier comprises an organosilane or at least two organosilanes which are different from each other, wherein the organosilane or one of the at least two organosilanes which are different from each other is (3-Glycidyloxypropyl)-trimethoxysilane; a functional constituent; and a protective layer to protect the functional constituent. In a preferred embodiment, the functional constituent reacts with the epoxy group of the organosilane to bind the functional constituent via the organosilane covalently to the surface of the solid carrier.

In a more preferred embodiment, the hydrophobic organosilane is selected from the group consisting of benzyl-triethoxysilane, (3-Glycidyl-oxypropyl)-trimethoxysilane and n-Octyl-triethoxysilane.

In a further most preferred embodiment, the organosilane is (3-Glycidyl-oxypropyl)-trimethoxysilane (GPTMS).

In a further embodiment, the composition comprises a solid carrier, wherein the surface of the solid carrier comprises at least three organosilanes, wherein at least two of the at least three organosilanes are different from each other.

In a further embodiment, the composition comprises a solid carrier, wherein the surface of the solid carrier comprises at least four organosilanes, wherein at least two or at least three of the at least four organosilanes are different from each other.

In one embodiment, the solid carrier is selected from the group of organic particles, inorganic particles, organic-inorganic particles, self-assembled organic particles, silica particles, gold particles, magnetic particles and titanium particles and is preferably an inorganic particle, more preferably a silica particle, even more preferably a silica nanoparticle (SNP). The particle size is usually measured by measuring the diameter of the particles. In case the solid carrier is a monodisperse particle, the size is usually between 1000 μm and 1 nm, preferably between 100 μm and 10 nm, particularly between 50 μm and 50 nm, more particularly between 1 μm and 100 nm. In case the solid carrier is a polydisperse particle, the size is usually between 1000 μm and 1 nm, preferably between 100 μm and 10 nm, particularly between 50 μm and 50 nm.

Usually monodisperse particles or polydisperse particles, preferably monodisperse particles are used as solid carrier in the present invention. In a preferred embodiment, the monodisperse particles are spherical monodisperse particles. In a further preferred embodiment, the polydisperse particles are non-spherical polydisperse particles.

In a further embodiment, the composition further comprising a linker. A linker usually connects the organosilane or the at least two organosilanes comprised by the surface of the solid carrier with the functional constituent, e.g., by covalently binding the organosilane or the at least two organosilanes comprised by the surface of the solid carrier and the functional constituent. Covalent binding of the organosilane or the at least two organosilanes comprised by the surface of the solid carrier and the functional constituent can alternatively be obtained by an organosilane comprised by the surface of the solid carrier which carries a functional binding group such as an epoxy group. Such an organosilane is, e.g., (3-Glycidyl-oxypropyl)-trimethoxysilane (GPTMS). In a preferred embodiment, the functional constituent is immobilized on the solid carrier by a linker, more preferably the functional constituent is immobilized on the solid carrier by a linker, wherein the linker binds covalently the organosilane or the at least two organosilanes comprised by the surface of the solid carrier and the functional constituent.

In a preferred embodiment, the linker is a bi-functional cross-linker, more preferably a bi-functional cross-linker selected from the group consisting of glutaraldehyde, disuccinimidyl tartrate, bis[sulfosuccinimidyl]suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils, sulfhydryl-reactive 2-pyridyldithiol, BSOCOES (Bis[2-(succinimidooxycarbonyloxy)ethyl]sulfone), DSP (Dithiobis[succinimidyl]propionate]), DTSSP (3,3′-Dithiobis[sulfosuccinimidyl]propionate]), DTBP (Dimethyl 3,3′-dithiobispropionimidate-2 HCl), DST (Disuccinimidyl tartarate), Sulfo-LC-SMPT (4-Sulfosuccinimidyl-6-methyl-a-(2-pyridyldithio)toluamido]hexanoate)), SPDP (N-Succinimidyl 3-(2-pyridyldithio)-propionate), LC-SPDP (Succinimidyl 6-(3-[2-pyridyldithio]-propionamido)hexanoate), SMPT (4-Succinimidyloxycarbonyl-methyl-a-[2-pyridyldithio]toluene), DPDPB (1,4-Di-[3′-(2′-pyridyldithio)-propionamido]butane), DTME (Dithio-bismaleimidoethane), BMDB (1,4 bismaleimidyl-2,3-dihydroxybutane). More preferably said bi-functional cross-linker is selected from glutaraldehyde, disuccinimidyl tartrate, disuccinimidyl suberate, bis[sulfosuccinimidyl] suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulthydrils (e.g. sulfhydryl-reactive 2-pyridyldithio). Most preferred is glutaraldehyde as linker.

In one embodiment, the functional constituent is immobilized on the solid carrier. In one embodiment, the functional constituent is immobilized on the solid carrier in random orientation. In one embodiment, the functional constituent is non-covalently bound to the surface of the solid carrier. Non-covalent binding includes p-p (aromatic) interactions, van der Waals interactions, H-bonding interactions, and ionic interactions. In a preferred embodiment, the functional constituent is covalently bound to the surface of the solid carrier. In a more preferred embodiment, the functional constituent is covalently bound to the surface of the solid carrier i) via an organosilane carrying a functional binding group such as an epoxy group or ii) via a linker.

In one embodiment, the size ratio of solid carrier to functional constituents is such that it allows binding of between 10 to 10000, preferably of between 50 to 5000, more preferably of between 100 to 1000 functional proteins per particle.

In one embodiment, the protective layer is formed by building blocks, wherein as building blocks structural building blocks and protective building blocks are used to form the protective layer. Preferably the structural building blocks are precursors of inorganic silica, capable of forming 4 covalent bonds in the layer formed and the protective building blocks are organosilanes, preferably the structural building blocks are tetravalent organosilanes, and the protective building blocks are trivalent organosilanes.

As building blocks for the protective layer usually structural building blocks and protective building blocks are used to build the protective layer.

Preferably the protective layer is formed by building blocks, wherein as building blocks structural building blocks and protective building blocks are used to form the protective layer, wherein the structural building blocks are precursors of inorganic silica, capable of forming 4 covalent bonds in the layer formed and the protective building blocks are organosilanes. Structural building blocks which can be used are, e.g., tetraethylorthosilicate (TEOS). Protective building blocks which can be used are, e.g., 3-Aminopropyltriethoxysilane (APTES), n-Propyltriethyoxysilane (PTES), Isobutyltriethoxysilane (IBTES), Hydroxymethyltriethoxysilane (HTMEOS), Benzyltriethoxysilane (BTES), Ureidopropyltriethoxysilane (UPTES), Carboxyethyltriethoxysilane (CETES).

Structural building blocks are usually precursors of inorganic silica, capable of forming 4 covalent bonds in the layer formed. Protective building blocks are usually organosilanes, bearing an organic moiety endowed with the ability to interact with the functional constituents (e.g., enzyme). Preferred structural building blocks are tetravalent silanes, in particular tetra-alkoxy-silanes. Preferred protective building blocks are trivalent silanes, in particular tri-alkoxy-silanes e.g. 3-Aminopropyltriethoxysilane (APTES), n-Propyltriethyoxysilane (PTES), Isobutyltriethoxysilane (IBTES), Hydroxymethyltriethoxysilane (HTMEOS), Benzyltriethoxysilane (BTES), Ureidopropyltriethoxysilane (UPTES), Carboxyethyltriethoxysilane (CETES). Most preferred structural building blocks are mixtures of tetravalent silanes and trivalent silanes, in particular, mixtures of tetra-alkoxy-silanes and tri-alkoxy-silanes. Particular preferred structural building blocks are selected from the group consisting of tetraethylorthosilicate, tetra-(2-hydroxyethyl)silane, and tetramethylorthosilicate. Particular preferred protective building blocks are selected from the group consisting of carboxyethylsilanetriol, benzylsilanes, propylsilanes, isobutylsilanes, n-octylsilanes, hydroxysilanes, bis(2-hydroxyethyl)-3-aminopropylsilanes, aminopropylsilanes, ureidopropylsilanes, (N-Acetylglycyl)-3-aminopropylsilanes, in particular, selected from benzyltriethoxysilane, propyltriethoxysilane, isobutyltriethoxysilane, n-octyltriethoxysilane, hydroxymethyltriethoxysilane, bis(2-hydroxyethyl)-3-aminopropyltriethoxysilane, 3-Aminopropyltriethoxysilane, ureidopropyltriethoxysllane, (N-Acetylglycyl)-3-aminopropyltriethoxysilane, or selected from benzyltrimethoxysflane, propyltrimethoxysilane, isobutylimethoxysilane, n-octyltrimethoxysilane, hydroxyrnethyltrimethoxysilane, bis(2-hydroxyethyl)-3-aminopropyltrimethoxysilane, arninopropyltrimethoxysilane, ureidopropyltrimethoxysilane (N-Acetylglycyl)-3-aminopropyltrimethoxysilane or selected from benzyltrihydroxyethoxysilane, propyltrihydroxyethoxysilane, isobutyltrihydroxyethoxysilane, n-octyltrihydroxyethoxysilane, hydroxymefilyltrihydroxyethoxysilane, bis(2-hydroxyethyl)-3-aminopropyltrihydroxyethoxysilane, aminopropyltrihydroxyethoxysilane, Ureidopropyltrihydroxyethoxysilane (N-Acetylglycyl)-3-aminopropyltrihydroxymethoxysilane.

A particular preferred building block is TEOS as structural building block and APTES and/or hydroxymethyltriethoxysilane, preferably APTES as protective building block. In particular, TEOS as structural building block and APTES as protective building block are used to build the protective layer. The reaction is usually carried out for a time period of between 0.5 to 10 hours, preferably between 1 and 5 hours, more preferably between 1 and 2 hours, preferably in aqueous solution and preferably at a temperature of about 5 to about 15° C. or at about 10° C. The formation of the protective layer can be stopped by actively stopping the polycondensation reaction, e.g, by removing the non-reacted building blocks, e.g., by a washing step or by self-stopping of the polycondensation reaction caused by a limited amount of building blocks.

In some embodiments the protective layer has a defined thickness of about 1 to about 100 nm, preferably about 1 to about 50 mm, more preferably about 1 to about 30 nm, even more preferably about 1 to about 25 nm, in particular, about 1 to about 20 nm, more particularly, about 1 to about 15 nm. In a preferred embodiment, the protective layer has a thickness of about 1 to about 30 nm, even more preferably about 1 to about 25 nm, in particular, about 1 to about 20 nm, more particularly, about 1 to about 15 nm. The protective layer is usually porous and the pore size is between 1 and 100 nm, preferably between 1 and 20 nm.

The protective layer thickness can be measured, by using a microscope such as scanning electron microscope (SEM), transmission electron microscopy (TEM), scanning probe microscopy (SPM), light scattering methods or by ellipsometry.

In a preferred embodiment, the functional constituent is a protein or a fragment thereof or RNA which has catalytic activity enzyme, more preferably an enzyme or a fragment thereof, an antibody or a fragment thereof, or RNA which has catalytic activity enzyme, even more preferably an enzyme or a fragment thereof, most preferably an enzyme or a fragment thereof selected from the group consisting of oxidoreductases, transferases, hydrolases, lyases, isomerases or ligases. Particular preferred is a hydrolase, more particular a lipase, even more particular a Candida antarctica lipase B (CALB).

In one embodiment, the protective layer embeds between 10% and 100%, preferably between 20% and 100%, more preferably between 30% and 100%, even more preferably between 50% and 100%, in particular, between 70% and 100% of the functional constituent.

In a further aspect, the present invention provides a composition comprising a solid carrier, wherein the surface of the solid carrier comprises an organosilane selected from the group consisting of hydrophobic organosilanes and hydrophilic organosilanes. In one embodiment, the hydrophilic organosilanes are selected from the group consisting of organosilanes carrying a functional polar group selected from an alcohol, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxvethoxvsilane, ureidopropyltrihydroxyethoxysilane; a functional constituent; and a protective layer to protect the functional constituent. Solid carrier, functional constituent and protective layer are as described above.

In one embodiment, the hydrophilic organosilanes are selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, and aminopropyltrihydroxvethoxvsilane, ureidopropyltrihydroxyethoxysilane. Solid carrier, functional constituent and protective layer are as described above.

In a further aspect, the present invention provides a composition comprising a solid carrier, wherein the surface of the solid carrier comprises a first organosilane and a second organosilane, wherein the second organosilane is different from the first organosilane; a functional constituent; and a protective layer to protect the functional constituent. Solid carrier, organosilane, functional constituent and protective layer are as described above.

In a further aspect, the present invention provides a method of producing a composition, the composition comprising a solid carrier, wherein the surface of the solid carrier comprises an organosilane with the proviso that the organosilane is not an aminosilane, or wherein the surface of the solid carrier comprises at least two organosilanes which are different from each other; a functional constituent; and a protective layer to protect the functional constituent, the method comprising the following steps:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) optionally re-suspending the solid carrier comprising the functional constituent protected by the protective layer in an organic solvent; and
(g) isolating the solid carrier comprising the functional constituent protected by the protective layer from the organic solvent suspension.

The composition of the present invention is usually produced in a reaction vessel like a reactor.

Suspension of the solid carrier in step a) of the present method can be, e.g., in an aqueous solution like water or aqueous buffer. In one embodiment, the aqueous solution is suspended in step a) in a solvent different from the solvent used in step f). In one embodiment, the solid carrier is suspended in step a) in a polar protic solvent, preferably water or buffer, and in step f) the solid carrier comprising the functional constituent protected by the protective layer is re-suspended in an organic solvent selected from the group consisting of organic polar aprotic solvents and organic non-polar solvents. In a preferred embodiment, the solid carrier is suspended in an aqueous solution in step a), more preferably in water or aqueous buffer, even more preferably in aqueous buffer. Buffers which can be used in the method of the present invention are phosphate, piperazine-N,N′-bis(2-ethanesulfonic acid), 2-Hydroxy-3-morpholinopropanesulfonic acid, N,N-bis[2-hydroxyethyl]-2-aminoethanesulfonic acid), (3-(N-morpholino)propanesulfonic acid), 2-[[1,3-dihydroxy-2-(hydroxymethyl)propan-2-yl]amino]ethanesulfonic acid, 4-(2-hydroxyethyl)-1-piperazineethanesulfonic acid), 3-(N,N-Bis[2-hydroxyethyl]amino)-2-hydroxypropanesulfonic acid, N,N-Bis(2-hydroxyethyl)-3-amino-2-hydroxypropanesulfonic acid, N-[Tris(hydroxymethyl)methyl]glycine, Diglycine, 4-(2-Hydroxyethyl)-1-piperazinepropanesulfonic acid, N,N-Bis(2-hydroxyethyl)glycine, N-[Tris(hydroxymethyl)methyl]-3-aminopropanesulfonic acid, N-(1,1-Dimethyl-2-hydroxyethyl)-3-amino-2-hydroxypropanesulfonic acid. Preferably a phosphate buffer is used.

The modification of the surface in step b) of the present method is usually carried out by adding the organosilanes or the at least two organosilanes or by adding the organosilanes or the at least two organosilanes in solution using, e.g., aqueous solutions or a 50% water/ethanol solutions as solvent. Organosilanes to be used for the method are as described above.

The immobilization of a functional constituent on the solid carrier in step c) of the present method is usually carried out by adding a solution of the functional constituent to the suspension of the solid carrier with the modified surface obtained after step b). Optionally a linker can be added to the suspension of the solid carrier with the modified surface obtained after step b) prior to adding a solution of the functional constituent to the suspension of the solid carrier. In a preferred embodiment the immobilization of a functional constituent on the solid carrier is carried out by providing a suspension of the solid carrier and adding a solution of the functional constituent, wherein the suspension with the added solution of the functional constituent is incubated to allow the functional constituent, e.g, the enzyme to bind on the surface of the solid carrier. Solid carrier to be used for the method are as described above.

The formation of the protective layer according to step (d) of the present method is usually carried out by forming the respective protective layer with building blocks, wherein the building blocks build the protective layer in a polycondensation reaction. The polycondensation is usually effected in solution, preferably in aqueous solution. The polycondensation is usually effected in the suspension of the solid carrier comprising the functional constituent protected by the protective layer. Polycondensation can be easily controlled and stopped if appropriate, allowing that a defined thickness of the protective layer is achieved. As building blocks for the protective layer, the ones described above can be used.

In one embodiment, the polarity of the solid carrier comprising the functional constituent protected by the protective layer isolated from the suspension in step (e) may be modified prior to step (f). Modification can occur by incubating the particles with an non-polar organosilane. During this step the final diameter of the particle will not change. Thus in one embodiment the method comprises the following steps:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) re-suspending the solid carrier comprising the functional constituent protected by the protective layer in an organic solvent; and
(g) isolating the solid carrier comprising the functional constituent protected by the protective layer from the organic solvent suspension, wherein the solid carrier comprising the functional constituent protected by the protective layer isolated from the suspension in step (e) is modified prior to step (f), preferably modified by incubating the solid carrier comprising the functional constituent protected by the protective layer isolated from the suspension in step (e) with an non-polar organosilane prior to re-suspending the solid carrier comprising the functional constituent protected by the protective layer in an organic solvent in step (f).

In a preferred embodiment, the method comprises the following steps:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) re-suspending the solid carrier comprising the functional constituent protected by the protective layer in an organic solvent, wherein the organic solvent is an organic non-polar solvent, preferably an organic non-polar solvent selected from the group consisting of ethyl-acetate, diethyl-ether, methyl-ethyl-ketone, pentane, hexane, heptane, cyclohexane, toluene, benzene and nitrobenzene, more preferably selected from the group consisting of toluene, benzene and heptane, even more preferably heptane; and
(g) isolating the solid carrier comprising the functional constituent protected by the protective layer from the organic solvent suspension, wherein the solid carrier comprising the functional constituent protected by the protective layer isolated from the suspension in step (e) is modified prior to step (f), preferably modified by incubating the solid carrier comprising the functional constituent protected by the protective layer isolated from the suspension in step (e) with an non-polar organosilane prior to re-suspending the solid carrier comprising the functional constituent protected by the protective layer in an organic solvent in step (f).

The non-polar organosilane used to incubate the particles prior to step (f) is usually selected from the group consisting of octyltriethoxysilane, benzyltriethoxysilane and butyltriethoxysilane, and is preferably octyltriethoxysilane. The solid carrier comprising the functional constituent protected by the protective layer isolated from the suspension in step (d) is usually incubated with an non-polar organosilane prior to step (f) between 0.1 to 5 hours, preferably between 0.5 to 2 hours.

The solid carrier comprising the functional constituent protected by the protective layer is usually isolated from the suspension in step (e) of the present method by centrifugation. The solid carrier is collected as a pellet and the supernatant is discarded.

The solid carrier comprising the functional constituent protected by the protective layer is usually optionally re-suspended in an organic solvent in step (f) of the present method by pipetting up and down at least 10 times. Usually, the re-suspended particles are incubated in the organic solvent between 5 to 48 hours, preferably 10 to 20 hours, preferably at a constant temperature between 2 to 25° C., more preferably at a constant temperature between 15 to 25° C., even more preferably at a constant temperature of around 20° C. In a preferred embodiment, the organic solvent is selected from the group consisting of organic non-polar solvents and organic polar aprotic solvents, in particular, the organic solvent is selected from the group consisting of organic non-polar solvents and organic polar aprotic solvents, wherein the organic non-polar solvent is selected from the group consisting of ethyl-acetate, diethyl-ether, methyl-ethyl-ketone, pentane, hexane, heptane, cyclohexane, toluene, benzene and nitrobenzene and wherein the organic polar aprotic solvent is selected from the group consisting of acetone, acetonitrile, tetrahydrofurane, dimethylformamide, and pyridine, more particular an organic solvent selected from the group consisting of acetone, benzene, toluene, acetonitrile, pyridine and heptane, even more particular an organic solvent selected from the group consisting of acetone and heptane. More preferably, the organic solvent is an organic polar aprotic solvent, even more preferably, an organic polar aprotic solvent selected from the group consisting of acetone, acetonitrile, tetrahydrofurane, dimethylformamide, and pyridine, with acetone being most preferred. Equally more preferably, the organic solvent is an organic non-polar solvent, even more preferably, an organic non-polar solvent selected from the group consisting toluene, benzene and heptane, with heptane being most preferred.

The solid carrier comprising the functional constituent protected by the protective layer is usually isolated from the organic solvent suspension in step (g) of the present method by centrifugation.

Optionally, the method further comprises the step (h) drying the solid carrier comprising the functional constituent protected by the protective layer to remove the organic solvent. The solid carrier is usually dried by rotary evaporation under mild conditions or drying with a speed-vac system.

In a further aspect, the present invention provides a composition comprising a solid carrier, a functional constituent and a protective layer to protect the functional constituent obtainable by the method as described supra. Solid carrier, organosilane, functional constituent and protective layer are as described supra.

In one embodiment, the present invention provides the use of the composition in a catalytic process, in particular, the use of the composition in a catalytic process, wherein an esterification reaction is catalyzed by the composition. In particular, it is possible to use the composition of the invention in a catalytic process wherein during the process the composition is subject to at least one of a pH different from the optimal pH of the functional constituent, in particular, such that the pH value differs at least by +/−0.5 pH units and/or up to +/−5 pH units from the pH optimal for the functional constituent and/or to chemical stresses; and/or to biological stresses; and/or to solvents; and/or to physical stress; and/or to elevated temperatures, which exceed the optimal temperature for the functional constituent by at least 5° C.; and/or up to 60° C., particularly by 50° C., particularly by 40° C. higher, particularly by 30° C., particularly by 20° C., particularly by 10° C.; and/or to reduced temperatures, which deviate from the optimal temperature for the functional constituent by at least 5° C.; and/or up to 60° C.

### EXAMPLES

### Example 1

Modification of the Surface of the Silica Particles with APTES (Comparative Example) and Benzyl-Triethoxysilane

### CalB Immobilization:

Modification of the surface using APTES as organosilane according to WO2015/014888: Silica particles (Supsil Premium, diameter: 250 nm) in a volume of 15 mL water and with a concentration of 20 mg/mL were incubated with 2.6×10⁻⁴ mmol (60.9 μL) of APTES at 20° C. for 90 min with 400 rpm mixing. Afterwards, the particles suspension was washed 3 times in nanopure water, and the particles were resuspended in nanopure water. CalB immobilization was carried out following the procedure disclosed in Example 1 of WO2015/014888. Glutaraldehyde was used as bifunctional crosslinker

### Modification of the Surface Using APTES and Benzyl-Triethoxysilane According to Invention:

Silica particles (Supsil Premium, diameter: 250 nm) in a volume of 15 mL and with a concentration of 20 mg/mL were incubated with 2.6×10⁻⁴ mmol (60.9 μL) of APTES and 2.6×10⁻⁴ mmol (69.3 μL) of benzyl-triethoxysilane 96% at 20° C. for 90 min with 400 rpm mixing. Afterwards, the particles suspension was washed 3 times in nanopure water, and the particles were resuspended in nanopure water. CalB immobilization was carried out following the procedure disclosed in Example 1 of WO2015/014888. Glutaraldehyde was used as bifunctional crosslinker.

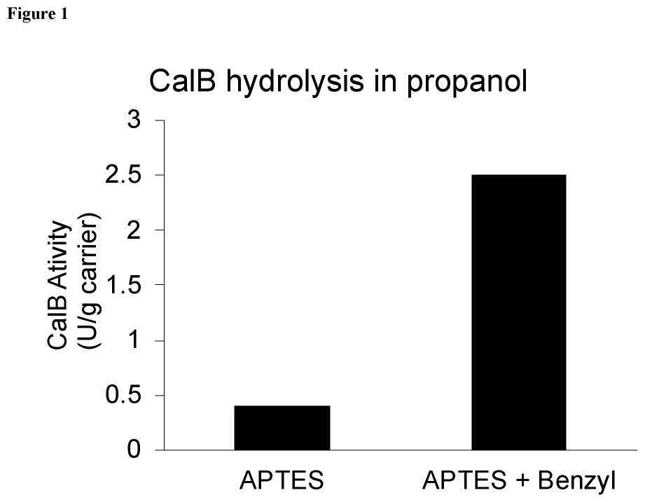

As shown in FIG. 1, the activity of the CalB immobilized on particles modified with APTES and benzyl-triethoxysilane was 6 times higher than the activity recovered in the particles modified with APTES only.

### Shielding of the Immobilized CalB

APTES and TEOS were used for the total shielding of the immobilized enzyme. After two washing steps in water, the SNPs with the modified surface were reacted during 30 minutes with the linker glutaraldehyde (to allow the further immobilization of the enzyme), at a final concentration of 1 g/L.

After two washing steps in water, the resulting SNPs were re-suspended in a KPi (potassium phosphate) buffer (pH 6, 10 mM) at a final concentration of 10 mg/mL and incubated for 1 hour at 20° C. with the enzyme, CALB, (6 mg/ml) under magnetic stirring at 400 rpm.

The protection of the enzyme immobilized on SNPs was carried out by incubating the produced enzyme-immobilized SNPs with a mixture of silane building blocks that self-assembled around the enzyme and underwent a polycondensation reaction that created a protecting layer around the enzyme. The polycondensation reaction also occurred at the bare surface of the SNPs allowing the attachment of this layer at the surface of the SNPs. To that end, enzyme-immobilized SNPs (20 mL; 10 mg/ml) were first reacted at 20° C. under stirring at 400 rpm with 356 μl of TEOS. After 1 hour of reaction, 71 μl of APTES were added and the protective layer was allowed to grow at 10° C. for 150 minutes. Samples of SNPs were collected by centrifugation. Particles were washed twice in acetone, resuspended in acetone and incubated for 12 hours at 20° C. A control sample of shielded CalB was washed twice in buffer and incubated for 12 hours at 20° C. in KPi buffer.

After incubation, the particles which were incubated in acetone were collected and dried by means of a rotary evaporator, and stored at 4° C. The catalytic activity of the enzyme prior to collection and after incubation in acetone was measured using an activity assay with a chromogenic artificial substrate which is the 4-nitrophenyl butyrate (NPB). In brief, the NPB is hydrolyzed into p-nitrophenol, which can be measured spectrophotometrically at 415 nm. The enzymatic activity was measured as μmol of p-nitrophenol produced per minute. Enzyme activity of the immobilized enzyme after incubation in acetone was five times higher than the activity of the particles which were incubated in KPi buffer.

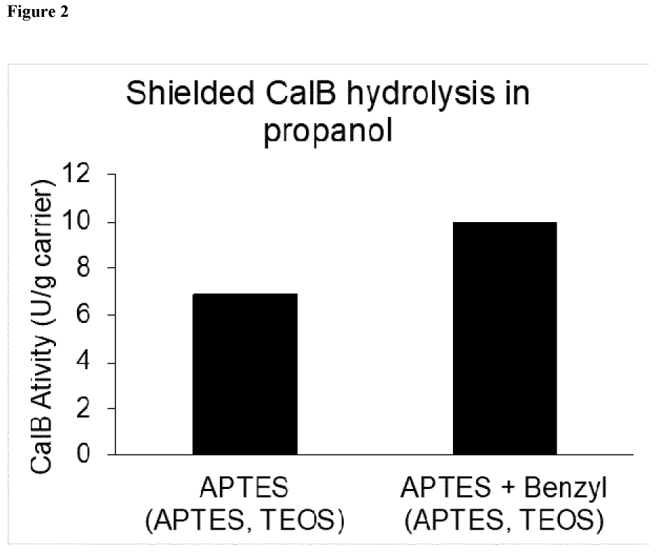

The obtained organosilica layers were comparable. They measured 10.0 nm for the carrier modified with APTES and 9.5 nm for the carrier modified with APTES and benzyl-triethoxysilane. The activities of the CalB immobilized on a carrier modified with APTES only or with APTES and benzyl-triethoxysilane and shielded with a layer of APTES and TEOS were 7 U/g carrier and 10 U/g carrier, respectively, as can be seen from FIG. 2.

### Example 2

Modification of the Surface of the Silica Particles with APTES (Comparative Example) and (3-Glycidyl-oxypropyl)-trimethoxysilane and n-Octyl-triethoxysilane

Silica particles (Supsil Premium, diameter: 250 nm) were resuspended in an ethanol/water solution (50/50 (v/v)) at a final concentration of 25 mg/mL in a final volume of 1 mL. A volume of 18 μL of 2 M NaOH were added. The surface of the particles was modified with different molar ratios of (3-glycidyl-oxypropyl)-trimethoxysilane (GPTMS) and n-octyl-triethoxysilane (octyl). In more details, the mmol of silanes added are the following: GPTMS/octyl 1/0 (1.8×10⁻⁵ mmol/0); GPTMS/octyl 1/0.25 (1.8×10⁻⁵ mmol/4.5×10⁻⁶ (mmol); GPTMS/octyl 1/0.5 (1.8×10⁻⁵ mmol/8.9×10⁻⁶ mmol); GPTMS/octyl 1/0.75 (1.8×10⁻⁵ mmol/1.3×10⁻⁵ mmol); GPTMS/octyl 1/1 (1.8×10⁻⁵ mmol/1.8×10⁻⁵ mmol) and GPTMS/octyl 0/1 (0/1.8×10⁻⁵ mmol); GPTMS/octyl 0.25/1 (4.5×10⁻⁶ mmol/1.8×10⁻⁵ mmol); GPTMS/octyl 0.5/1 (8.6×10⁻⁶ mmol/1.8×10⁻⁵ mmol); GPTMS/octyl 0.75/1 (1.3×10⁻⁵ mmol/1.8×10⁻⁵ mmol). Particle's suspensions were incubated for 60 min at 20° C. with 1300 rpm shaking. The enzyme crosslinking on the particles takes place through the epoxy group of the GPTMS. Afterwards, the particles suspensions were washed three times in water and resuspended in buffer and incubated for 1 hour at 20° C. with 1300 rpm shaking. As control, a sample of amino modified particles (APTES) as described in Example 1 above (comparative example according to WO2015/014888) was taken as reference. When APTES modified particles are used, the enzyme binding takes place through glutaraldehyde crosslinking as described in Example 1. CalB was used as model enzyme. After enzyme immobilization, samples were washed, and an activity assay was performed according to the assay described in example 1.

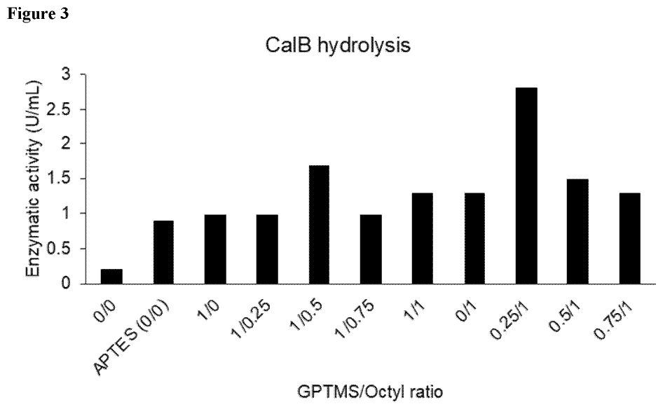

As shown in FIG. 3, the enzymatic activity recovered in particles modified with GPTMS and Octyl with molar ratio 4.5×10-6 mmol/1.8×10-5 mmol was 2.8 U/mL of particles suspension, meaning 14 times higher than the activity recovered in particles not modified and 3 times higher than the activity recovered on particles modified with APTES.

### Example 3

Modification of the Surface of the Silica Particles with (3-Glycidyloxypropyl)-trimethoxysilane

Silica particles (Supsil Premium, diameter: 250 nm) were resuspended in an ethanol/water solution (50/50 (v/v)) at a final concentration of 10 mg/mL in a final volume of 5 mL. A volume of 54 μL of 2 M NaOH was added. The surface of the particles was modified with 5.3×10⁻⁴ mmol of (3-glycidyl-oxypropyl)-trimethoxysilane (GPTMS). Particle's suspensions were incubated for 60 min at 20° C. with 1300 rpm shaking. Afterwards, the particles suspensions were washed three times in water and resuspended in buffer and incubated for 18 hours at 20° C. with 1300 rpm shaking in presence of 0.25 mg/mL of CalB. After enzyme immobilization, samples were washed, and an activity assay was performed according to the assay described in example 1.

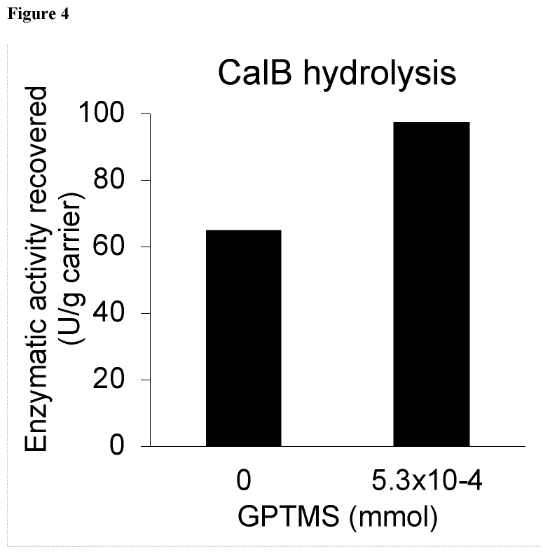

As shown in FIG. 4, the enzymatic activity recovered in particles modified with 5.3×10⁻⁴ mmol of GPTMS was 97.5 U/g_{carrier}, while the activity recovered on particles not treated was 65 U/g_{carrier} meaning 1.5 times lower than the activity recovered in particles modified with GPTMS.

1. A composition comprising:
a solid carrier, wherein a surface of the solid carrier comprises an organosilane with a proviso that the organosilane is not an aminosilane, or wherein the surface of the solid carrier comprises at least two organosilanes which are different from each other;
a functional constituent; and
a protective layer to protect the functional constituent.

2. The composition according to claim 1, wherein the surface of the solid carrier comprises two organosilanes, wherein the first organosilane is a hydrophobic organosilane or a hydrophilic organosilane and the second organosilane is a hydrophilic organosilane, wherein the second organosilane is different from the first organosilane.

3. The composition according to claim 1, wherein the surface of the solid carrier comprises two organosilanes, wherein the first organosilane is a hydrophobic organosilane and the second organosilane is a hydrophilic organosilane.

4. The composition according to claim 1, wherein the surface of the solid carrier comprises two organosilanes, wherein the first organosilane is a hydrophobic organosilane and the second organosilane is a hydrophobic organosilane, wherein the second hydrophobic organosilane is different from the first hydrophobic organosilane.

5. The composition according to claim 2, wherein the hydrophilic organosilane is selected from the group consisting of organosilanes carrying a functional polar group selected from an alcohol, an amine, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane.

6. The composition according to claim 2, wherein the hydrophilic organosilane is selected from the group consisting of organosilanes carrying a functional polar group selected from an alcohol, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyl-trihydroxyethoxysilane, (N-Acetylglycyl)-3-aminopropyltrihydroxymethoxysilane, aminopropyltrihydroxyethoxysilane, and ureidopropyltrihydroxyethoxysilane.

7. The composition according to claim 2, wherein the hydrophobic organosilane is selected from the group consisting of benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane, and n-octyl-triethoxysilane.

8. The composition according to claim 2, wherein the first organosilane is a hydrophobic organosilane selected from the group consisting of benzyl-triethoxysilane, (3-Glycidyl-oxypropyl)-trimethoxysilane and n-Octyl-triethoxysilane.

9. The composition according to claim 2, wherein the first organosilane is benzyl-triethoxysilane.

10. The composition according to claim 3, wherein the first organosilane is benzyl-triethoxysilane and the second organosilane is 3-aminopropyltriethoxysilane (APTES).

11. The composition according to claim 4, wherein the first organosilane is (3-Glycidyl-oxypropyl)-trimethoxysilane and the second organosilane is n-Octyl-triethoxysilane.

12. The composition according to claim 1, wherein the surface of the solid carrier comprises (3-Glycidyl-oxypropyl)-trimethoxysilane.

13. The composition according to claim 1, wherein the functional constituent is a protein or a fragment thereof, preferably an enzyme or a fragment thereof.

14. The composition according to claim 1, wherein the solid carrier is a silica particle.

15. A method of producing a composition, the composition comprising a solid carrier, wherein a surface of the solid carrier comprises an organosilane with a proviso that the organosilane is not an aminosilane, or wherein the surface of the solid carrier comprises at least two organosilanes which are different from each other; a functional constituent; and a protective layer to protect the functional constituent, the method comprising:
(a) providing a suspension of a solid carrier;
(b) modifying the surface of the solid carrier by adding an organosilane to the suspension with the proviso that the organosilane added is not an aminosilane; or modifying the surface of the solid carrier by adding at least two organosilanes which are different from each other to the suspension;
(c) immobilizing a functional constituent on the solid carrier;
(d) forming a protective layer on the surface of the solid carrier to protect the functional constituent immobilized on the solid carrier;
(e) isolating the solid carrier comprising the functional constituent protected by the protective layer from the suspension;
(f) optionally re-suspending the solid carrier comprising the functional constituent protected by the protective layer in an organic solvent; and
(g) isolating the solid carrier comprising the functional constituent protected by the protective layer from the organic solvent suspension.

16. The composition according to claim 3, wherein the hydrophilic organosilane is selected from the group consisting of organosilanes carrying a functional polar group selected from an alcohol, an amine, a carboxylate, a thiol, a thioether, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyltrihydroxyethoxysilane, (N-Acetylglycyl)-3-aminopropyltrihydroxymethoxysilane, aminopropyltrihydroxyethoxysilane, and ureidopropyltrihydroxyethoxysilane.

17. The composition according to claim 3, wherein the hydrophilic organosilane is selected from the group consisting of organosilanes carrying a functional polar group selected from an alcohol, a carboxylate, a thiol, a thither, a guanidinium, an amide, and selected from the group consisting of hydroxymethyltriethoxysilane, bis(2-hydroxyethyl)-3-aminopropyltriethoxysilane, ureidopropyltriethoxysilane, (N-Acetylglycyl)-3-aminopropyltriethoxysilane, hydroxymethyltrimethoxysilane, bis(2-hydroxyethyl)-3-aminopropyltrimethoxysilane, ureidopropyltrimethoxysilane, (N-Acetylglycyl)-3-aminopropyltrimethoxysilane, hydroxymethyltrihydroxyethoxysilane, bis(2-hydroxyethyl)-3-aminopropyl-trihydroxyethoxysilane, (N-Acetylglycyl)-3-aminopropyltrihydroxymethoxysilane, aminopropyltrihydroxyethoxysilane, and ureidopropyltrihydroxyethoxysilane.

18. The composition according to claim 3, wherein the hydrophobic organosilane is selected from the group consisting of benzyl-triethoxysilane, propyltriethoxysilane, isobutyltriethoxysilane, benzyltrimethoxysilane, propyltrimethoxysilane, isobutyltrimethoxysilane, n-octyltrimethoxysilane, benzyltrihydroxyethoxysilane, propyltrihydroxyethoxysilane, isobutyltrihydroxyethoxysilane, n-octyltrihydroxyethoxysilane, (3-Glycidyl-oxypropyl)-trimethoxysilane, and n-octyl-triethoxysilane.

19. The composition according to claim 3, wherein the first organosilane is a hydrophobic organosilane selected from the group consisting of benzyl-triethoxysilane, (3-Glycidyl-oxypropyl)-trimethoxysilane and n-Octyl-triethoxysilane.

20. The composition according to claim 4, wherein the first organosilane is a hydrophobic organosilane selected from the group consisting of benzyl-triethoxysilane, (3-Glycidyl-oxypropyl)-trimethoxysilane and n-Octyl-triethoxysilane.

21. The composition according to claim 3, wherein the first organosilane is benzyl-triethoxysilane.

22. The composition according to claim 4, wherein the first organosilane is benzyl-triethoxysilane.
